(19) United States
(12) Patent Application Publication
(10) Pub. No.: US 2025/0270722 A1
(43) Pub. Date: Aug. 28, 2025

(54) METHOD FOR CONVERTING CARBON DIOXIDE INTO SNG OR LNG AND STORING HYDROGEN

(71) Applicant: SAIPEM S.P.A., Milano (IT)

(72) Inventors: Matteo BERRA, Milano (IT); Lorenzo BRUNO, Milano (IT)

(73) Assignee: SAIPEM S.P.A., Milano (IT)

(21) Appl. No.: 18/879,042

(22) PCT Filed: Jun. 30, 2023
(86) PCT No.: PCT/IB2023/056840

§ 371 Date: Dec. 26, 2024

(30) Foreign Application Priority Data
Jun. 30, 2022 (IT) 102022000013888

(51) Int. Cl. C25B 15/08; B01D 53/26; C07C 1/12; F25J 1/00

(52) U.S. Cl. CPC C25B 15/081; B01D 53/265; C07C 1/12; F25J 1/001; F25J 1/0022; B01D 2256/16; B01D 2256/245; B01D 2257/80; F25J 2205/86; F25J 2260/80

(57) ABSTRACT

Methods are for storing electricity and producing liquefied natural gas (LNG) or synthetic natural (SNG) and using carbon dioxide and for producing electricity, natural gas (NG) or SNG. The methods involve, starting from a water flow, producing an oxygen gas flow and a hydrogen gas flow by electrolysis in an electrolytic cell. A first hydrogen gas flow portion and a second hydrogen gas flow portion are obtained. The first hydrogen gas flow portion is allocated to a methanation step in the presence of carbon dioxide gas. A condensed recirculation water vapor flow is obtained to be allocated to the methanation step and performing methanation. The second hydrogen gas flow portion is allocated to a cooling and liquefaction step. A liquid hydrogen flow is obtained, which is stored in a liquid hydrogen tank.

### CROSS-REFERENCE TO RELATED APPLICATION

The present application is a National Stage Application of PCT International Application No.: PCT/IB2023/056840 filed on Jun. 30, 2023, which claims priority to Italian Application 102022000013888, filed in the Italian Patent Office on Jun. 30, 2022, the disclosure of which is incorporated herein by reference in its entirety.

### FIELD OF THE INVENTION

The present invention applies to the field of use of sequestered carbon dioxide and production of SNG and/or liquefied natural gas (LNG).

### BACKGROUND OF THE INVENTION

Methanation reactors require an almost continuous supply: in fact, frequent switching on and off reduces the life of the catalyst and heat exchange trains, as well as requiring rather long times to develop the thermal profiles which ensure the correct conversion of the reagents.

This is obviated by producing and consuming hydrogen, in methanation, even in periods of energy scarcity, trying to reduce the hydrogen needs of the reactors by bringing them to turndown.

Alternatively, it is possible to store that hydrogen not to be produced by using energy, in the form of compressed gas or in liquid form, much more suitable for large storage.

To date, the prior art hydrogen liquefaction requires 10 to 13 KWh/kg (source DOE), where the lowest values are obtained using low-revolution volumetric compressors (efficiency 90%, as reported in “Large scale hydrogen liquefaction under the aspect of economic viability”).

Since the lower calorific value of hydrogen amounts to about 33.3 kWh/kg, it is noted that liquefaction is particularly onerous.

As for magnetic liquefaction, it appears suitable only for small-scale applications.

Attempts to obtain greater energy efficiency in liquefaction have led to replacing the pre-cooling of hydrogen by means of liquid nitrogen, with a refrigeration cycle employing a particular refrigerant mixture, consisting of light hydrocarbons and nitrogen, but there is a risk that some components of said mixture can freeze in the cryogenic process.

Again, since Sabatier's reaction is very exothermic, the temperature control in methanation reactors is problematic, and typically requires to recycle large amounts of reaction products entering into the first reactor, employing a recirculation compressor working at high temperatures, therefore technologically challenging.

Prior art document CN109943373 describes a process comprising the hydrolysis of water and the use of carbon dioxide in methanation, in which a control of the temperature of the reactors by means of vapor injection is implemented.

Prior art document JP 2020 024065 describes a method for continuously supplying hydrogen to a hydrogen liquefaction system.

### SUMMARY OF THE INVENTION

The inventors of the present patent application have developed a process in which a part of hydrogen produced by hydrolysis of water is sent to methanation reactors which, together with carbon dioxide, produce LNG or SNG, while another part is cooled and possibly condensed by virtue of a hydrogen cooling system, to store it in liquid and/or cryo-compressed form.

### OBJECT OF THE INVENTION

In a first object, the present invention describes a method for storing electricity and producing liquefied natural gas (LNG) or synthetic natural gas (SNG) and using carbon dioxide.

According to a preferred aspect, such stored energy is excess energy as compared to needs.

In a second object, the present invention describes a method for producing electricity, natural gas (NG) or synthetic natural gas (SNG).

A method is described as a whole, which, as a function of the availability of electricity, allows storing electricity and producing liquefied natural gas (LNG) or synthetic natural gas (SNG), and using carbon dioxide and producing electricity, natural gas (NG) or synthetic or substitute natural gas (SNG).

The present invention further relates to a plant for storing available excess electricity and producing liquefied natural gas or synthetic natural gas and using carbon dioxide, where said plant, under conditions of electricity need, can also produce power, in the form of electricity.

### BRIEF DESCRIPTION OF THE DRAWINGS

FIG. 1 shows the part of the plant operating method referred to as the storage step, as provided by a first embodiment of the plant.

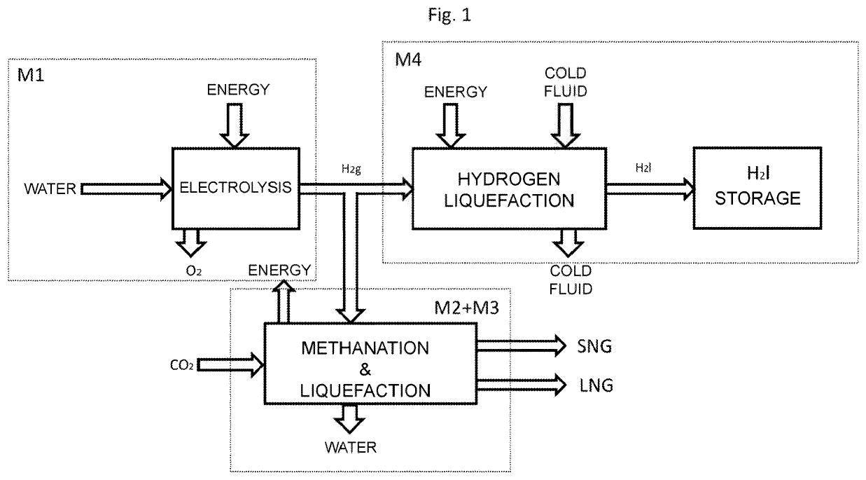

FIG. 2 shows the part of the plant operating method referred to as the continuity step, as provided by a first embodiment of the plant.

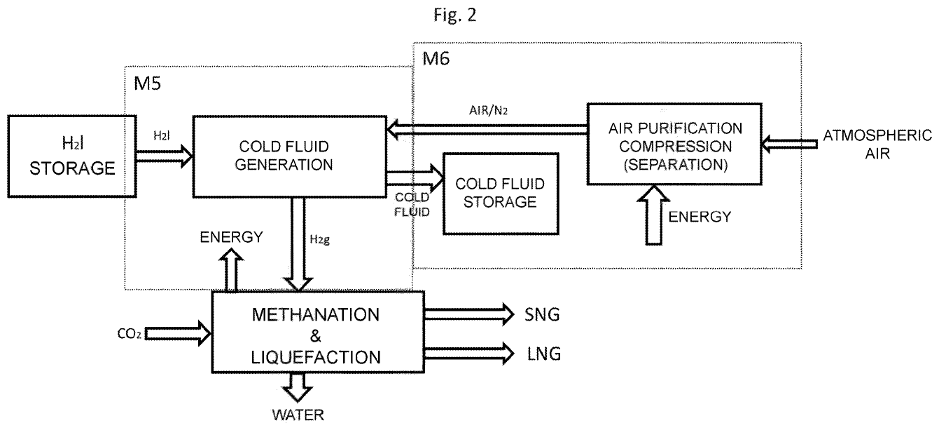

FIG. 3 shows the part of the plant operating method referred to as the storage step, as provided by a second embodiment of the plant.

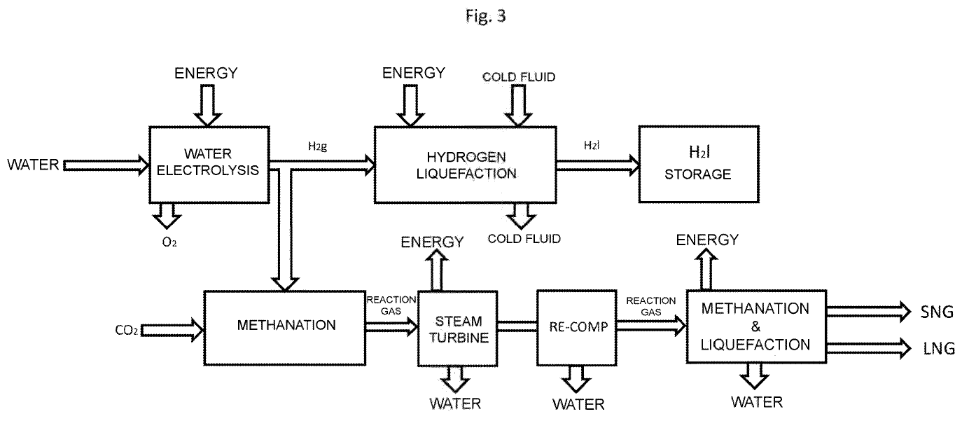

FIG. 4 shows the part of the plant operating method referred to as the continuity step, as provided by a second embodiment of the plant.

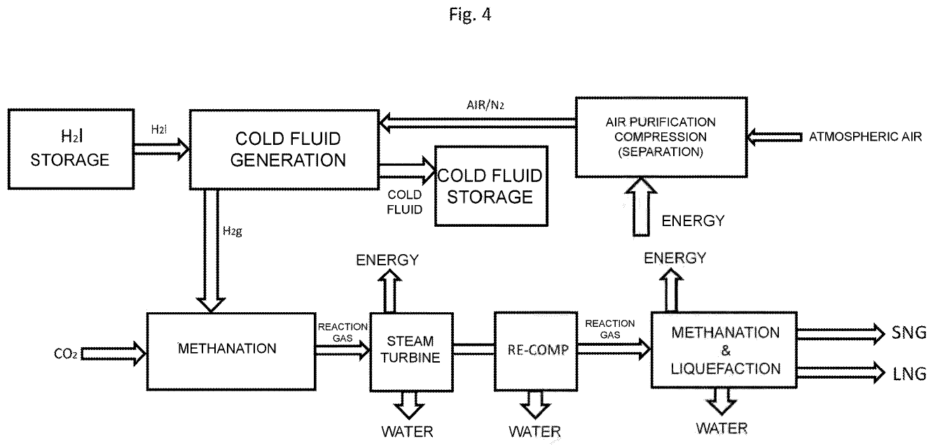

FIG. 5 shows an embodiment of the storage step A) of the method of the present invention.

FIG. 6 shows an embodiment of the chemical continuity step B) of the method of the present invention.

### DETAILED DESCRIPTION OF THE INVENTION

For the purposes of the present invention, the heat exchanges conducted in the exchangers (E1, E2, E3, E4, En) are conducted with external fluids and preferably represented by air, water, etc.

The heat exchangers (indicated by “EXn”) instead allow the heat exchange between two flows inside a circuit or circuits described in the invention.

The method of the present invention comprises a step A), said energy storage step, and a step B), said chemical continuity step.

For the purposes of the present invention, the storage step A) is a step in which hydrogen gas is produced to be allocated to a methanation step together with a carbon dioxide flow for producing LNG or SNG, and hydrogen gas allocated partly for methanation and partly for storage in liquid form.

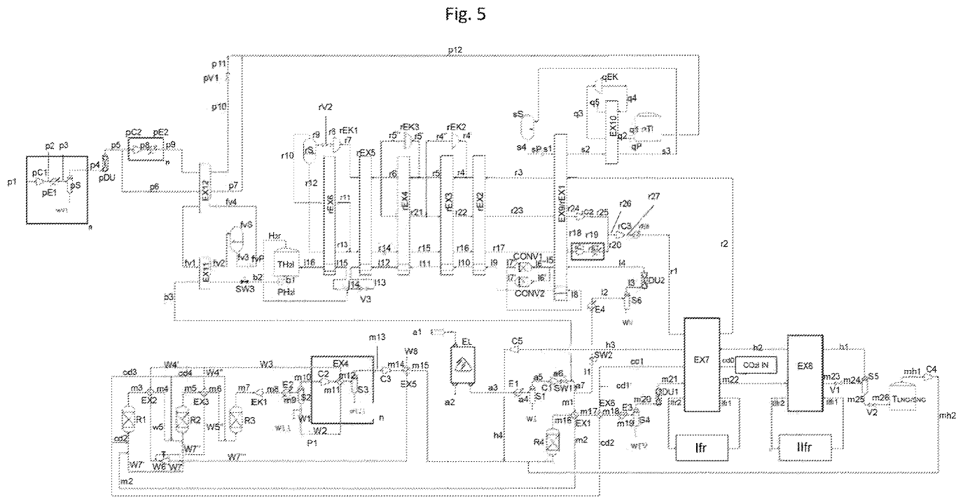

An embodiment of step A) of the present invention is diagrammatically depicted in FIG. 5, where the switch valves SW1 and SW2 are open, while SW3 is closed.

More in detail, the energy storage step A comprises the sub-steps of:
A1) starting from a water flow a1, producing an oxygen gas flow a2 and a hydrogen gas flow a3 by electrolysis in an electrolytic cell EL,
A2) obtaining a first hydrogen gas flow portion m1 and a second hydrogen gas flow portion 11,
A3) allocating said first hydrogen gas flow portion m1 to a methanation step and obtaining a condensed recirculation water vapor flow W1 to be allocated to said methanation step,
A4) allocating said second hydrogen gas flow portion 11 to a cooling and liquefaction step, thus obtaining a liquid hydrogen flow 116.

Therefore, for the purposes of the present invention, step A3) comprises the use of carbon dioxide gas, as will be described hereinafter.

In particular, the carbon dioxide gas flow used in the methanation step is obtained from a liquid CO₂ source (CO₂ IN in the figure).

More in particular, a liquid CO₂ flow cd0 is subjected to a first heating step, thus obtaining a first CO₂ gas flow cd1.

Said first CO₂ gas flow cd1 is subjected to a second heating step so as to obtain a methanation CO₂ flow cd2.

According to an aspect of the present invention, a further CO₂ gas flow cd1′ obtained from a CO₂ gas source can be added to said first CO₂ gas flow cd1.

For the purposes of the present invention, the electrolysis sub-step A1) is conducted using electricity, possibly from renewable sources, such as solar energy.

For this purpose, the water flow a1 is fed to the electrolytic cell EL at appropriate pressure, for example at pressures up to 30 barg or even up to 80 barg.

The oxygen gas flow obtained a2 can be allocated to other purposes or liquefied and possibly marketed.

According to a preferred aspect of the present invention, the electricity employed for the hydrolysis sub-step A1) is electricity available in excess.

The term “available in excess” means an amount of electricity available in the electrical network which is not required by the system.

For the purposes of the present invention, sub-step A2) comprises the further sub-steps of:
A2a) subjecting said hydrogen gas flow a3 to cooling, thus obtaining a cooled hydrogen gas flow a4,
A2b) in a first separator S1, separating a first portion of condensed water vapor wI from said cooled hydrogen gas flow a4, thus obtaining a partially dehydrated hydrogen flow a5,
A2c) subjecting said partially dehydrated hydrogen flow a5 to compression in a first compressor C1, thus obtaining a compressed hydrogen flow a6,
A2d) separating a first hydrogen gas flow portion m1 and a second hydrogen gas flow portion 11.

For the purposes of the present invention, sub-step A3) comprises the further sub-steps of:
A3a) subjecting said first hydrogen gas flow portion m1 to heating in a first heat exchanger EX1, thus obtaining a methanation hydrogen flow m2,
A3b) subjecting said methanation hydrogen flow m2 to methanation in the presence of the methanation carbon dioxide flow cd2 and obtaining a methanation product flow m7,
A3c) obtaining a flow of partially dehydrated methanation products m10 and a condensed recirculation water vapor flow W1,
A3d) obtaining a flow of further dehydrated methanation products m13,
A3e) subjecting said flow of further dehydrated methanation products m13 to a further methanation step, thus obtaining a flow of further methanation products m16,
A3f) subjecting said flow of further methanation products m16 to cooling, thus obtaining a flow of cooled products of further methanation m19,
A3g) subjecting said flow of cooled products of further methanation m19 to dehydration, thus obtaining a flow of dehydrated products of further methanation m21,
A3h) subjecting said flow of dehydrated products of further methanation m21 to further cooling, thus obtaining a two-phase flow of further methanation products m23,
A3i) obtaining a recirculation hydrogen flow h1 and a final liquid flow of methanation products m26.

For the purposes of the present invention, said step A3b) comprises the still further steps of:
A3b1) subjecting said methanation hydrogen flow m2 to a first methanation step in a first methanation reactor R1, thus obtaining a first methanation product m3,
A3b2) cooling, thus obtaining a first cooled methanation product m4,
A3b3) subjecting said first cooled methanation product m4 to a second methanation step in a second methanation reactor R2, thus obtaining a second methanation product m5,
A3b4) cooling, thus obtaining a second cooled methanation product m6,
A3b5) subjecting said second cooled methanation product m6 to a third methanation step in a third methanation reactor R3, thus obtaining the methanation product flow m7.

For the purposes of the present invention, in the second methanation step A3b3) a first methanation CO₂ portion cd3 separated from the methanation CO₂ flow cd2 is employed.

For the purposes of the present invention, in the third methanation step A3b5) a second methanation CO₂ portion cd4 separated from the first methanation CO₂ portion cd3 is employed.

For the purposes of the present invention, step A3c) comprises the still further steps of:
A3c1) expanding the methanation product flow m7 in a first expander EK1, with possible power production, thus obtaining a flow of expanded methanation products m8,
A3c2) cooling in a second exchanger E2, thus obtaining a flow of expanded and cooled methanation products m9,
A3c3) separating a second condensed water vapor portion wII in a second separator S2, thus obtaining the flow of partially dehydrated methanation products m10.

According to an aspect of the present invention, a recirculation condensed water vapor flow W1 is separated from the second water vapor portion wII separated in step A3c3), which is allocated to the methanation step.

In particular, said recirculation water vapor flow W1 is allocated to the first and/or second methanation steps A3b3).

Advantageously, said control water vapor has the function of controlling the temperature of the methanation process.

According to a particular aspect of the present invention, said recirculation water vapor flow W1 is in sequence:
pumped from a first pump P1, thus obtaining a pumped recirculation water vapor flow W2,
heated in a fourth heat exchanger EX4, thus obtaining a pumped and heated water vapor flow W3,
divided into a first portion and a second portion of pumped and heated water vapor, W4′ and W4″, respectively, each of which is heated, thus obtaining a first portion and a second portion of pumped and further heated water vapor, W5′ and W5″, respectively, which are combined in the single pumped and further heated water vapor flow W6, which is expanded in the turbine Tl with power production, thus obtaining an expanded water vapor flow W7.

According to an aspect of the present invention, from said expanded water vapor flow W7 is obtained:
a first control vapor portion W7′ sent to the first methanation step A3b1),
a second control vapor portion W7″ sent to the second methanation step A3b3),
a third vapor portion W7′″ which is cooled, thus obtaining a heat exchange water flow W8.

According to such an aspect of the present invention, the heating of the first pumped and heated water vapor portion W4′ is conducted within the second heat exchanger EX2 by heat exchange with the first methanation product m3.

According to such an aspect of the present invention, the heating of the second pumped and heated water vapor portion W4″ is conducted within the third heat exchanger EX3 by heat exchange with the second methanation product m5.

For the purposes of the present invention, said step A3d) comprises the still further sub-steps of:
A3d1) subjecting said flow of partially dehydrated methanation products m10 to compression in a second compressor C2, thus obtaining a flow of partially dehydrated and compressed methanation products m11,
A3d2) cooling said flow of partially dehydrated and compressed methanation products m11, thus obtaining a flow of partially dehydrated, compressed and cooled methanation products m12,
A3d3) separating a third condensed water vapor flow wIII within a third separator S3, thus obtaining a flow of further dehydrated methanation products m13.

According to such an aspect, step A3d2) is conducted within a fourth heat exchanger EX4 for heat exchange with the pumped recirculation water vapor flow W2.

For the purposes of the present invention, step A3d) can be repeated once or more according to needs.

For the purposes of the present invention, before being subjected to the further methanation step A3e), said flow of further dehydrated methanation products m13 is subjected to the steps of:
compression in a third compressor C3, thus obtaining a flow of further dehydrated and compressed methanation products m14,
heating in a fifth heat exchanger EX5, thus obtaining a flow of further dehydrated, compressed and heated methanation products M15.

According to an aspect of the present invention, the heating step is conducted in the fifth heat exchanger EX5 by heat exchange with the third vapor portion W7′″.

For the purposes of the present invention, said step A3f) comprises the still further steps of:
A3f1) subjecting said flow of further methanation products m16 to a first partial cooling, thus obtaining a flow of further methanation products at a first partial cooling level m17,
A3f2) subjecting said flow of further methanation products at a first partial cooling level m17 to a second partial cooling, thus obtaining a flow of further methanation products at a second partial cooling level m18,
A3f3) subjecting said flow of further methanation products at a second partial cooling level m18 to a third partial cooling in a third exchanger E3, thus obtaining a flow of cooled products of further methanation m19.

For the purposes of the present invention, said first cooling step A3f1) is conducted within the first heat exchanger EX1 by heat exchange with the first hydrogen gas flow portion m1 allocated for methanation (step A3a) described above).

For the purposes of the present invention, said second cooling step A3f2) is conducted within a sixth heat exchanger EX6 by heat exchange with the CO₂ gas flow cd1, thus obtaining a methanation CO₂ flow cd2.

For the purposes of the present invention, said step A3g) comprises the still further steps of:
A3g1) separating a fourth condensed vapor portion wIV in a fourth separator S4, thus obtaining a flow of cooled and partially dehydrated products of further methanation m20,
A3g2) dehydrating said flow of cooled and partially dehydrated products of further methanation m20 in a first dehydration unit DU1, thus obtaining a flow of dehydrated products of further methanation m21.

For the purposes of the present invention, said step A3h) comprises the still further steps of:
A3h1) subjecting said flow of dehydrated products of further methanation m21 to a first cooling, thus obtaining a flow of cooled and dehydrated products of further methanation at a first cooling level m22,
A3h2) subjecting said flow of cooled and dehydrated products of further methanation at a first cooling level m22 to a second cooling in an eighth heat exchanger EX8, thus obtaining a two-phase flow of further methanation products m23.

For the purposes of the present invention, said step A3h1) is conducted within a seventh heat exchanger EX7 by heat exchange with the liquid CO₂ flow cd0, thus obtaining a first CO₂ gas flow cd1.

For the purposes of the present invention, said step A3i) comprises the still further steps of:
A3i1) expanding said two-phase flow of further methanation products m23 by means of a first expansion valve V1, thus obtaining an expanded two-phase flow m24,
A3i2) separating from said expanded two-phase flow m24 a recirculation hydrogen flow h1 from the head of a fifth separator and a methanation product-enriched flow m25,
A3i3) further expanding said methanation product-enriched flow m25 in a second expansion valve V2, thus obtaining a final liquid flow of methanation products m26.

Said final liquid flow of methanation products m26 is then stored in a tank of methanation products TLNG/SNG.

For the purposes of the present invention, a methane- and hydrogen-rich gas flow mh1 can be withdrawn from the methanation product tank TLNG/SNG.

In an aspect of the present invention, said methane- and hydrogen-rich gas flow mh1 is compressed in a fourth compressor C4, thus obtaining a compressed methane- and hydrogen-rich gas flow mh2, which is sent to the further methanation step A3e) in the fourth reactor R4.

As described above, a recirculation hydrogen flow h1 is also obtained from step A3i2).

For the purposes of the present invention, said recirculation hydrogen flow h1 is subjected to the steps of:
first heating, thus obtaining a heated recirculation hydrogen flow h2,
second heating, thus obtaining a further heated recirculation hydrogen flow h3,
compression in a fifth compressor C5, thus obtaining a compressed recirculation hydrogen flow h4.

According to such an embodiment of the invention, said compressed recirculation hydrogen flow h4 is sent to the further methanation step A3e) in the fourth reactor R4.

For the purposes of the present invention, in step A3h1) and A3i2) described above, the heat exchange in the seventh heat exchanger EX7 is also conducted by heat exchange with the heated recirculation hydrogen flow h2.

For the purposes of the present invention, the heat exchange step further involves a first refrigerant fluid flow Ifr1, circulating in a first refrigerant fluid circuit (Ifr in the figure) and which gives its frigories in the heat exchanges within the seventh heat exchanger EX7, thus obtaining a heated first refrigerant fluid flow Ifr2.

Within the first refrigerant fluid cycle, said heated flow is then cooled according to methods known in the art (and not depicted in the figures), so as to provide a flow to be involved in the further heat exchanges within the seventh heat exchanger EX7.

According to an aspect of the present invention, such a first refrigerant fluid is a fluid selected from the group comprising: propane, carbon dioxide or commercially available refrigerants.

In particular, for the purposes of the present invention, steps A3h2) and A3i2) in the eighth heat exchanger EX8 described above are conducted by heat exchange between said flow of cooled and dehydrated products of further methanation at a first cooling level m22 and the recirculation hydrogen flow h1.

For the purposes of the present invention, said heat exchange further involves a second refrigerant fluid flow IIfr1, circulating in a second refrigerant fluid circuit (IIfr in the figure) and which gives its frigories in the heat exchanges within the eighth heat exchanger EX8, thus obtaining a heated second refrigerant fluid flow IIfr2.

According to an aspect of the present invention, said second refrigerant fluid is a fluid selected from the group comprising: ethylene, methane, ethane, nitrogen, or mixtures thereof.

As described above, in step A4) a second hydrogen gas flow portion 11 is allocated to a cooling and liquefaction step, thus obtaining liquid hydrogen.

For the purposes of the present invention, step A4) with which the second hydrogen gas flow portion 11 is allocated to cooling and liquefaction, thus obtaining a liquid hydrogen flow 116, comprises the sub-steps of:
A4a) dehydrating said second hydrogen gas flow portion 11 and obtaining a second dehydrated hydrogen gas portion 14,
A4b) cooling said second dehydrated hydrogen gas portion 14 and obtaining a liquid hydrogen flow 116, which is stored in a liquid hydrogen tank TH2l.

For the purposes of the present invention, said step A4a) comprises the further sub-steps of:
A4a1) cooling said second hydrogen gas flow portion 11 in a fourth exchanger E4, thus obtaining a cooled second hydrogen gas flow portion 12,
A4a2) separating a fifth condensed water vapor portion wV in a sixth separator S6, thus obtaining a partially dehydrated flow of second hydrogen gas portion 13,
A4a3) further dehydrating said partially dehydrated from of second hydrogen gas portion 13 in a second dehydration unit DU2, thus obtaining a second dehydrated hydrogen gas portion 14.

For the purposes of the present invention, step A4b) comprises one or more heat exchanges with one or more flows of a hydrogen refrigerating fluid (hereinafter abbreviated as “frh”) circulating within a cycle of the hydrogen refrigerating fluid, said flows being characterized by different temperatures and/or pressures.

To this end, said heat exchanges can be conducted in a ninth heat exchanger EX9 and in further heat exchangers, as will be described hereinafter.

According to an aspect of the present invention, the flows within said hydrogen refrigerating fluid cycle can be involved in one or more separation, lamination, expansion steps with possible power production, mixing therebetween, compression and heat exchange with one or more external fluids and/or with one or more further refrigerant fluids, where said heat exchanges can be direct or indirect.

For example, the hydrogen refrigerating fluid cycle can be a Claude cycle, as will be described hereinafter by way of non-limiting example.

According to an aspect of the present invention, the hydrogen refrigerating fluid can in turn be cooled by heat exchange with a further hydrogen refrigerating fluid flow, circulating within a circuit of a further hydrogen refrigerating fluid.

For the purposes of the present invention, the further hydrogen refrigerating fluid is liquid air or liquid nitrogen.

In particular, in a step I) a first further pumped refrigerant fluid flow s1 carries out a heat exchange with which it gives its frigories to the hydrogen refrigerating fluid, thus obtaining a second heated flow of further refrigerant fluid s2.

For example, such a heat exchange can be conducted within the ninth heat exchanger EX9.

Said second heated flow of further hydrogen refrigerating fluid s2 is then subjected to a step II) in which it is cooled, thus obtaining a cooled flow of further hydrogen refrigerating fluid s3, which in a step III) is sent to a collection tank sS; from said collection tank sS in a step IV), a liquid flow of further hydrogen refrigerating fluid s4 can be withdrawn, which in a step V) is pumped by a pump of further hydrogen refrigerating fluid sP, thus obtaining the first hydrogen refrigerating fluid flow s1.

As for step II) of cooling the second fluid flow of further hydrogen refrigerating fluid s2 described above, this is conducted involving a liquid air flow.

In particular, step II) is conducted within a tenth heat exchanger EX10 by heat exchange with a pumped liquid air flow q2, thus obtaining a heated liquid air flow q3.

In turn, said pumped liquid air flow q2 is obtained pumping a liquid air flow q1 by means of a liquid air pump qP.

The initial liquid air (or liquid nitrogen) flow q1 is obtained from a tank qTl in which the liquid air is stored, as will be described hereinafter in relation to step B) of the method of the invention.

According to an embodiment of the present invention, said heated liquid air flow q3 can then be expanded in a liquid air expander qEK, with possible power production, thus obtaining an expanded air flow q4, which can be further heated by heat exchange within the tenth heat exchanger EX10, thus obtaining a heated air flow q5 which can be released into the atmosphere.

According to an embodiment of the present invention, the hydrogen refrigerating fluid can also carry out a heat exchange with one or more further flows.

In this respect, the hydrogen refrigerating fluid can be involved in the heat exchanges within the seventh heat exchanger EX7 by cooling.

Therefore, step A) of the method of the present invention allows obtaining:
oxygen gas,
LNG or SNG,
liquid hydrogen,
power production; in particular, power can be produced:
in the first expander EK1,
in any expanders of the hydrogen refrigerating fluid circuit,
in the liquid air flow expander qEK.

However, the power production does not exceed the amount of energy absorbed by the network, in particular for step A1) of water hydrolysis; in fact, the energy produced is mainly used for the operation of the machines implementing the method.

Therefore, step A) of the method of the present invention allows employing:
excess electricity; in particular, such energy is used in step A1) of water electrolysis and, to a lesser extent, in the operation of the machines: compressors and pumps.

As for step B) of the method of the present invention, this comprises a sub-step B1), in which, starting from a liquid hydrogen storage, a continuous hydrogen gas flow b3 is obtained to be allocated to a methanation step, thus obtaining LNG or SNG.

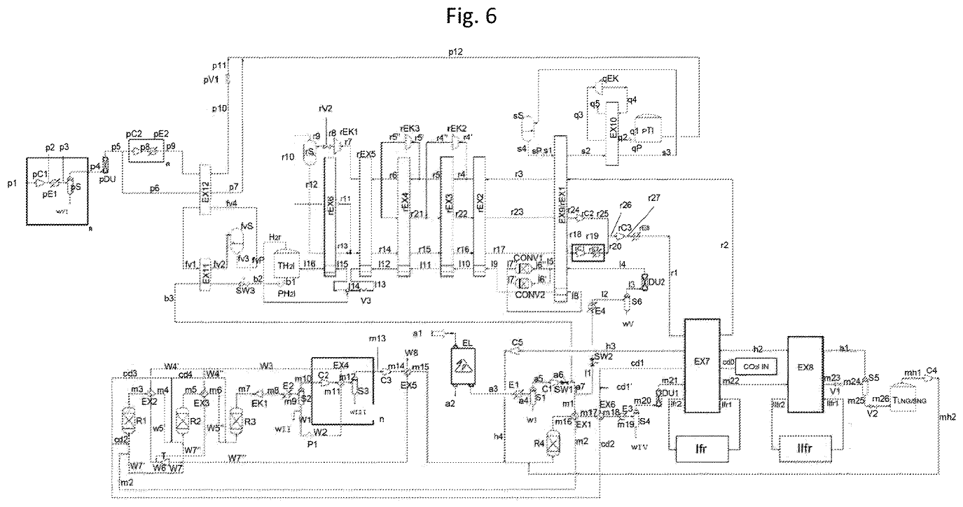

An embodiment of step B) of the present invention is schematically depicted in FIG. 6, where the switch valves SW1 and SW2 are closed, while SW3 is open.

Step B further comprises producing a liquid air storage.

More in particular, sub-step B1 comprises the further sub-steps of:
B1a) withdrawing a continuous liquid hydrogen flow b1 from a liquid hydrogen tank TH2l,
B1b) pumping said flow with a liquid hydrogen pump PH21, thus obtaining a pumped continuous liquid hydrogen flow b2,
B1c) heating said pumped continuous liquid hydrogen flow b2, thus obtaining a continuous hydrogen gas flow b3 and allocating it to said methanation step as described above.

When required, said continuous hydrogen gas flow b3 forms the first portion of said hydrogen gas flow m1.

As for step B1d), such heating is conducted within an eleventh heat exchanger EX11 by heat exchange with a first carrier fluid flow fv1 circulating within a carrier fluid cycle.

For the purposes of the present invention, said carrier fluid is nitrogen or oxygen-depleted air.

In particular, after heat exchange with the continuous expanded hydrogen flow b3, a second cooled carrier fluid flow fv2 is obtained which is collected in a carrier fluid tank fvS.

A third carrier fluid flow fv3 can be withdrawn therefrom, pumped by a pump of the carrier fluid fvP giving a fourth carrier fluid flow fv4, which is heated by heat exchange giving the first carrier fluid flow fv1.

In particular, the heating of the fourth carrier fluid flow fv4 is obtained in a twelfth heat exchanger EX12 for heat exchange with one or more heat exchange air flows allocated for producing a liquid air storage, as described hereinafter.

In particular, an initial atmospheric air flow p1 is subjected to the steps of:
compression and refrigeration, with possible separation of condensed water vapor,
dehydration,
thus obtaining a dehydrated air flow p5.

In particular, the initial atmospheric air flow p1 is compressed in a first atmospheric air compressor pC1, thus obtaining a compressed atmospheric air flow p2.

Such a compressed atmospheric air flow p2 is cooled in a first atmospheric air exchanger pE1, thus obtaining a compressed and cooled atmospheric air flow p3.

The condensed water vapor is separated within an atmospheric air separator pS, thus obtaining a sixth condensed water vapor portion wVI and a partially dehydrated atmospheric air flow p4.

The compression and refrigeration steps can be repeated as needed.

The partially dehydrated atmospheric air flow is then further dehydrated in an air dehydration unit pDU, thus obtaining the dehydrated air flow p5.

A portion of dehydrated air p6 is separated from the dehydrated air flow p5.

Such a dehydrated air portion p6 is involved in the heat exchange step conducted in the twelfth heat exchanger EX12 with the fourth carrier fluid flow fv4.

As for the dehydrated air flow p5, this is subjected to compression and cooling steps to obtain a heat exchange-dehydrated air flow p9, which carries out a further heat exchange in the twelfth heat exchanger Ex12 with the fourth carrier fluid flow fv4.

In particular, the dehydrated air flow p5 is compressed in a dehydrated air compressor pC2, thus obtaining a compressed dehydrated air flow p8, which is further cooled in a dehydrated air heat exchanger pE2, thus obtaining the heat exchange-dehydrated air flow p9.

The heat exchange-dehydrated air flow p9 and the dehydrated air portion p6 are two heat exchange air flows as described above; in fact, both flows are cooled by heat exchange in the twelfth heat exchanger EX12 with the fourth carrier fluid flow fv4, from which a first liquid air flow p10 and a second liquid air flow p7 are obtained.

Said first liquid air flow p10 is then depressurized by a first air valve pV1, thus obtaining a depressurized flow of the first liquid air flow p11.

The depressurized flow of the first liquid air flow p11 and the second liquid air flow p7 are combined in a single liquid air flow p12, which is sent to a liquid air tank pT1.

For the purposes of the present invention, the continuous liquid hydrogen flow b1 of step B1a) is withdrawn from a liquid hydrogen tank TH21 containing liquid hydrogen stored during step A4) described above.

Then, the methanation step is conducted on a hydrogen gas flow obtained from liquid hydrogen stored during step A4) described above, to be employed in step B) under circumstances of electricity need or, in other words, under circumstances in which electricity (with which to conduct the hydrolysis step A1)) is not available in excess.

For the purposes of the present invention, the tank in which liquid air pT1 is stored in step B) is the tank from which the initial liquid air flow q1 is withdrawn, which, when pumped, originates the pumped liquid air flow q2 involved in the heat exchange in the tenth heat exchanger EX10 of step A).

### Example of Step A4b

As described above, step A4b) comprises cooling and liquefying the second dehydrated hydrogen gas portion 14, thus obtaining a liquid hydrogen flow 116, which is thus stored in a liquid hydrogen tank TH21.

Such cooling can be conducted by virtue of the cycle of a hydrogen refrigerating fluid (abbreviated as “frh”) according to different modes known in the art.

One of the modes described below merely by way of example is the Claude cycle (referred to as the step C), which is shown in FIG. 5 for convenience.

In accordance with such a mode, the cooling is conducted by a first heat exchanger of the cycle rFH rEX1 (such an exchanger corresponds to the ninth heat exchanger EX9 described in step A above).

In accordance with such a mode, the dehydrated hydrogen gas flow 14 is subjected to the following steps:
C1) first cooling in a first heat exchanger of the cycle rFH rEX1, thus obtaining a hydrogen flow at a first cooling level 15,
C2) obtaining a first portion 16′ and a second portion 16″ of the hydrogen flow at a first cooling level, each of which is subjected to ortho/para allotropic conversion into a first and a second converter CONV1 and CONV2, respectively, thus obtaining converted first portion 17′ and second portion 17″, which are combined in the converted single flow 18,
C3) the single converted flow 18 is cooled by heat exchange and further converted within the special conversion section of the first exchanger of the cycle frh rEX1c, thus obtaining a hydrogen flow at a second cooling level 19,
C4) cooling and conversion in a second heat exchanger of the cycle frh rEX2, thus obtaining a hydrogen flow at a third cooling level 110,
C5) cooling and conversion in a third heat exchanger of the hydrogen refrigerating fluid cycle rEX3, thus obtaining a hydrogen flow at a third cooling level 111,
C6) cooling and conversion in a fourth heat exchanger of the cycle frh rEX4, thus obtaining a hydrogen flow at a fourth cooling level 112,
C7) cooling and conversion in a fifth heat exchanger of the cycle frh rEX5, thus obtaining a hydrogen flow at a fifth cooling level 113,
C8) expanding said hydrogen flow at a fifth cooling level 113 by means of a third expansion valve V3, thus obtaining an expanded hydrogen flow at a fifth cooling level 114,
C9) combining with a recirculation hydrogen flow H2r to give a single hydrogen flow 115,
C10) cooling and conversion in a sixth heat exchanger of the cycle rEX6 frh, thus obtaining a liquid hydrogen flow 116, which is stored in a liquid hydrogen tank TH21.

The recirculation hydrogen flow H2r is obtained from the head of such a tank TH21, which can be combined with the expanded hydrogen flow at the fifth cooling level 114 as described in step C9) above.

Each of the hydrogen flow cooling steps is conducted in the presence of an appropriate catalyst within an appropriate section of the heat exchanger, which converts the hydrogen from the ortho allotropic form to the para form, with resulting stabilization.

Therefore, each cooling step is also a conversion and stabilization step.

As for the hydrogen refrigerating fluid cycle, if represented by a Claude cycle, this can be described starting from a first hydrogen refrigerating fluid flow r1 (abbreviated as refrigerant fluid and indicated by reference sign “r”), which is cooled by heat exchange within the seventh heat exchanger EX7, thus obtaining a second refrigerant fluid flow r2.

The second flow frh r2 is cooled by heat exchange in the first exchanger of the cycle frh rEX1, thus obtaining a third flow frh r4.

Said third flow frh r3 is cooled in the second exchanger of the cycle frh rEX2, thus obtaining a fourth flow frh r4.

The fourth flow frh r4 is cooled in the third exchanger of the cycle frh rEX3, thus obtaining a fifth flow frh r5.

The fifth flow frh r5 is cooled in the fourth exchanger of the cycle frh rEX4, thus obtaining a sixth flow frh r6.

The sixth flow frh r6 is cooled in the fifth exchanger of the cycle frh rEX5, thus obtaining a seventh flow frh r7.

The seventh flow frh r7 is subjected to expansion in a first expander of the cycle frh rEK1, with possible power production, thus obtaining an eighth flow frh r8.

The eighth flow frh r8 is further expanded by a second valve of the cycle rfh rV2, thus obtaining a ninth flow frh r9.

Within a separator of the cycle frh rS, a twelfth liquid flow frh r12 and a tenth gaseous flow frh r10 are obtained from the ninth flow frh r9.

The tenth gas flow frh r10 is heated by heat exchange of the sixth heat exchanger of the cycle frh rEX6, thus obtaining an eleventh flow frh r11.

The twelfth flow r12 is heated by heat exchange of the sixth heat exchanger of the cycle frh rEX6, thus obtaining a thirteenth flow frh r13, to which an eleventh flow frh r11 is combined.

The thirteenth flow r13 is heated by heat exchange in the fifth heat exchanger frh rEX5, thus obtaining a fourteenth flow frh r14.

The fourteenth flow frh r14 is heated by heat exchange in the fourth heat exchanger rfh rEX4, thus obtaining a fifteenth flow r15.

The fifteenth flow frh r15 is heated by heat exchange in the third heat exchanger frh rEX3, thus obtaining a sixteenth flow frh r16.

The sixteenth flow r16 is heated by heat exchange in the second heat exchanger frh rEX2, thus obtaining a seventeenth flow frh r17.

The seventeenth flow frh r17 is heated by heat exchange in the first heat exchanger frh rEX1, thus obtaining an eighteenth flow frh r18.

The eighteenth flow frh r18 is subjected to a compression step in a first compressor of the cycle of frh rC1, thus obtaining a nineteenth flow frh r19, which is cooled in a seventh exchanger of the cycle frh rE7, thus obtaining a twentieth flow frh r20.

Optionally, said compression and cooling steps can be repeated if required.

According to a possible embodiment, a portion r4′ is separated from the fourth flow frh r4, which is expanded with possible power production in a second expander of the cycle frh rEK2, thus obtaining a further fourth flow frh r4″.

According to a possible embodiment, a portion r5′ is separated from the fifth flow frh r5, which is expanded with possible power production in a third expander of the cycle frh rEK3, thus obtaining a further fifth flow frh r5″.

Said further fifth flow frh r5″ is heated by heat exchange in the fourth heat exchanger of the cycle frh rEK4, thus obtaining a twenty-first flow frh r21.

The twenty-first flow frh r21 is heated by heat exchange in the third heat exchanger rEX3, thus obtaining a twenty-second flow frh r22.

The twenty-second flow r22 frh is heated by heat exchange in the second heat exchanger rEX2, thus obtaining a twenty-third flow r23 frh.

The twenty-third flow frh r23 is heated by heat exchange in the first heat exchanger rEX1, thus obtaining a twenty-fourth flow frh r24, which is compressed in a second compressor of the cycle frh rC2, thus obtaining a twenty-fifth flow frh r25.

Said twenty-fifth flow rfh r25 is combined with the twenty-seventh flow rfh r20, thus obtaining a twenty-sixth flow rfh r26, which is compressed in a third compressor of the cycle frh rC3, thus obtaining a twenty-seventh flow rfh r27, which is cooled in an eighth cooler rEX8, thus obtaining the first hydrogen refrigerating fluid flow r1.

The present invention further relates to a plant for storing available excess electricity and producing liquefied natural gas or synthetic natural gas and using carbon dioxide, where said plant, under conditions of electricity need, can also produce power, in the form of electricity.

In particular, such a plant comprises:
a module for producing hydrogen by water electrolysis (M1),
a module for methanation and optional production of power (M2),
a first and optionally a second refrigerant fluid cycle, for liquefying the methanation products (M3),
a module for liquefying hydrogen gas and producing power (M4),
a module for gasifying liquid hydrogen (M5),
a module for liquefying air (M6),
valves for allocating the hydrogen flow obtained by electrolysis to methanation or liquefaction, and for allocating the gasified hydrogen flow from liquefied hydrogen to methanation,
tanks for storing liquid hydrogen (TH21), liquid methanation products (TLNG SNG), liquid air (pT1),
For the purposes of the present invention, said module for producing hydrogen by electrolysis of water also produces oxygen.

For the purposes of the present invention, the module for producing hydrogen by electrolysis of water electricity available in excess, as described above.

According to an aspect of the present invention, said methanation module comprises one or more methanation reactors.

According to an aspect of the present invention, said methanation module comprises a system for dehydrating methanation products and recirculating at least part of the separated water to the methanation reactors themselves.

According to an aspect of the present invention, said methanation module employs gaseous or gasified carbon dioxide.

According to an aspect of the present invention, the module for liquefying hydrogen gas and producing power comprises a circuit of a further hydrogen refrigerating fluid.

According to an aspect of the present invention, the liquid hydrogen gasification module comprises a cycle of a carrier fluid.

According to an aspect of the present invention, said carrier fluid cycle is also part of the liquid air circuit.

For the purposes of the present invention, the described plant comprises valves in an open configuration for sending part of the hydrogen gas obtained by water electrolysis to the methanation module (M2) and part of the hydrogen gas obtained by water electrolysis to the hydrogen liquefaction module (M4), while the valve for sending the liquefied hydrogen to the hydrogen gas gasification module (M5) is closed.

The advantages offered by the present invention will become immediately apparent from the above description.

Firstly, the method described allows using the carbon dioxide sequestered, from the environment or from other sources (flue gas) for producing a combustible gas, which can be fed to the natural gas network.

As a further and not secondary advantage, the method described allows obtaining supply continuity of hydrogen and carbon dioxide to the methanation reactors (chemical continuity), ensuring process continuity.

This continuity allows avoiding shutting down the reactors in circumstances in which there is a lack of electricity to be used for the hydrolysis of water.

The method described, as a whole, allows stabilizing the electrical network, absorbing any excess electricity when available in excess, and storing it so as to make it available in moments of shortage.

The described method also allows the stabilization of the gas network, being able to partially supply the network by virtue of the vaporization of the liquefied natural gas obtained with the method of the invention.

The part of oxygen gas, LNG and/or SNG and hydrogen gas produced can possibly be marketed.

More specifically, the method of the invention optimizes the methanation step by operating a reaction control by virtue of the use of separate water vapor in the method itself.

Moreover, this also allows eliminating the reacted gas recycling compressor typically used in the methanation section and which, due to the severe operating conditions, forms a considerable technological commitment; this is achieved by virtue of the injection of water vapor to replace the recirculation of the reacted gases.

The method of the invention allows storing large amounts of hydrogen beyond the daily and/or seasonal availability of energy, limiting the amount of energy lost in hydrogen cooling, which means a greater capacity of conversion of CO₂ into SNG.

The method described is capable of offering a deferred storage system: the excess energy is transformed into SNG\LNG, in turn used by other electricity generation and/or transport systems.

1. A method for storing electricity and producing liquefied natural gas or synthetic natural gas and for using carbon dioxide, comprising the steps of:
A1) starting from a water flow, producing an oxygen gas flow and a hydrogen gas flow by electrolysis in an electrolytic cell,
A2) obtaining a first hydrogen gas flow portion and a second hydrogen gas flow portion,
A3) allocating said first hydrogen gas flow portion to a methanation step in the presence of carbon dioxide gas and obtaining a condensed recirculation water vapor flow to be allocated to said methanation step and performing methanation, and
A4) allocating said second hydrogen gas flow portion to a cooling and liquefaction step, thus obtaining a liquid hydrogen flow, which is stored in a liquid hydrogen tank.

2. The method according to claim 1, wherein step A1) is conducted using available excess electricity and/or electricity from renewable sources.

3. The method of claim 1, wherein in step A3) said carbon dioxide gas is obtained from liquid carbon dioxide.

4. The method according to claim 1, wherein Step A3) comprises the sub-steps of:
A3a) subjecting said first hydrogen gas flow portion to heating in a first heat exchanger, thus obtaining a methanation hydrogen flow,
A3b) subjecting said methanation hydrogen flow to methanation in the presence of a methanation carbon dioxide flow and obtaining a methanation product flow,
A3c) obtaining a flow of partially dehydrated methanation products and the condensed recirculation water vapor flow,
A3d) obtaining a flow of further dehydrated methanation products,
A3e) subjecting said flow of further dehydrated methanation products to a further methanation step, thus obtaining a flow of further methanation products,
A3f) subjecting said flow of further methanation products to cooling, thus obtaining a flow of cooled products of further methanation,
A3g) subjecting said flow of cooled products of further methanation to dehydration, thus obtaining a flow of dehydrated products of further methanation,
A3h) subjecting said flow of dehydrated products of further methanation to further cooling, thus obtaining a two-phase flow of further methanation products, and
A3i) obtaining a recirculation hydrogen flow and a final liquid flow of methanation products.

5. The method according to claim 4, wherein sub-step A3b) comprises the further sub-steps of:
A3b1) subjecting said methanation hydrogen flow to a first methanation step in a first methanation reactor, thus obtaining a first methanation product,
A3b2) cooling, thus obtaining a first cooled methanation product,
A3b3) subjecting said first cooled methanation product to a second methanation step in a second methanation reactor, thus obtaining a second methanation product,
A3b4) cooling, thus obtaining a second cooled methanation product, and
A3b5) subjecting said second cooled methanation product to a third methanation step in a third methanation reactor, thus obtaining the methanation product flow,
wherein in the second methanation step, a first methanation carbon dioxide portion separated from the methanation carbon dioxide flow is employed, and
wherein in the third methanation step, a second methanation carbon dioxide portion separated from the first methanation carbon dioxide portion is employed.

6. The method according to claim 4, wherein sub-step A3c) comprises the further sub-steps of:
A3c1) expanding the methanation product flow in a first expander, thus obtaining a flow of expanded methanation products,
A3c2) cooling in a second exchanger, thus obtaining a flow of expanded and cooled methanation products, and
A3c3) separating a second condensed water vapor portion in a second separator, thus obtaining the flow of partially dehydrated methanation products.

7. The method according to claim 6, wherein sub-step A3d) comprises the further sub-steps of:
A3d1) subjecting said flow of partially dehydrated methanation products to compression in a second compressor, thus obtaining a flow of partially dehydrated and compressed methanation products,
A3d2) cooling said flow of partially dehydrated and compressed methanation products, thus obtaining a flow of partially dehydrated, compressed and cooled methanation products, and
A3d3) separating a third condensed water vapor portion in a third separator, thus obtaining the flow of further dehydrated methanation products.

8. The method according to claim 7, wherein said condensed recirculation water vapor flow is employed in the first and/or second methanation steps.

9. The method according to claim 1, wherein step A4) comprises the sub-steps of:
A4a) dehydrating said second hydrogen gas flow portion and obtaining a second dehydrated hydrogen gas portion,
A4b) cooling said second dehydrated hydrogen gas portion and obtaining the liquid hydrogen flow, which is stored in the liquid hydrogen tank,
wherein sub-step A4b) comprises one or more heat exchanges with one or more hydrogen refrigerating fluid flows.

10. The method according to claim 9, wherein said one or more hydrogen refrigerating fluid flows are cooled by heat exchange with a further hydrogen refrigerating fluid flow, which circulates within a circuit of a further hydrogen refrigerating fluid and is liquid air or liquid nitrogen.

11. A method for producing electricity, natural gas (NG) or synthetic natural gas (SNG) comprising a step B1), wherein, starting from a liquid hydrogen storage, a continuous hydrogen gas flow is obtained, to be allocated to a methanation step, thus obtaining NG or SNG.

12. The method according to claim 11, wherein said step B1) comprises the sub-steps of:
B1a) withdrawing a continuous liquid hydrogen flow from a liquid hydrogen tank,
B1b) pumping said continuous liquid hydrogen flow with a liquid hydrogen pump, thus obtaining a pumped continuous liquid hydrogen flow, and
B1c) heating said pumped continuous liquid hydrogen flow, thus obtaining a continuous hydrogen gas flow, and allocating the continuous hydrogen gas flow to said methanation step.

13. The method according to claim 12, wherein sub-step B1d) is conducted by heat exchange with a first carrier fluid flow circulating within a carrier fluid cycle.

14. The method according to claim 13, wherein said carrier fluid flow performs a heat exchange with one or more heat exchange air flows for producing liquid air.

15. The method according to claim 14, comprising the steps of:
A1) starting from a water flow, producing an oxygen gas flow and a hydrogen gas flow by electrolysis in an electrolytic cell,
A2) obtaining a first hydrogen gas flow portion and a second hydrogen gas flow portion,
A3) allocating said first hydrogen gas flow portion to a methanation step in the presence of carbon dioxide gas and obtaining a condensed recirculation water vapor flow to be allocated to said methanation step and performing methanation, and
A4) allocating said second hydrogen gas flow portion to a cooling and liquefaction step, thus obtaining the liquid hydrogen flow, which is stored in the liquid hydrogen tank,
wherein the continuous liquid hydrogen flow of sub-step B1a) is withdrawn from the liquid hydrogen tank containing a liquid hydrogen flow stored during step A4) for storing electricity and producing liquefied natural gas (LNG) or synthetic natural gas (SNG) and for using carbon dioxide.

16. A method for storing electricity and producing liquefied natural gas (LNG) or synthetic natural gas and using carbon dioxide and for producing electricity, natural gas or synthetic natural gas, the method comprising the step of conducting the method of claim 1, or a method for producing electricity, natural gas or synthetic natural gas comprising a step B1), wherein, starting from a liquid hydrogen storage, a continuous hydrogen gas flow is obtained, to be allocated to a methanation step, thus obtaining natural gas or synthetic natural gas.

17. A plant for storing available excess electricity and producing liquefied natural gas or synthetic natural gas and using carbon dioxide, wherein said plant, under conditions of electricity need, is configured to produce electricity, said plant comprising:
a module for producing gaseous hydrogen by water electrolysis,
a module for methanation and optional production of power,
a first and optionally a second refrigerant fluid cycle, for liquefying methanation products,
a module for liquefying hydrogen gas and producing power,
a module for gasifying liquid hydrogen,
a module for liquefying air,
tanks for storing the liquid hydrogen, liquid methanation products, and liquid air, and
valves for allocating a hydrogen flow obtained by electrolysis to methanation or liquefaction and a valve for allocating a gasified hydrogen flow from liquefied hydrogen to methanation,
wherein said valves for allocating the hydrogen flow obtained by electrolysis to methanation or liquefaction are in an open configuration for sending part of the gaseous hydrogen produced by water electrolysis to the methanation module and part of the gaseous hydrogen produced by water electrolysis to the hydrogen liquefaction module, while the valve for sending the liquefied hydrogen to the hydrogen gas gasification module is closed, or vice versa.

18. The plant according to claim 17, wherein the module for methanation is configured for methanation and production of power, and wherein the plant further comprises a second refrigerant fluid cycle, for liquefying the methanation products.

19. The method according to claim 4, wherein sub-step A3c) comprises the further sub-steps of:
A3c1) expanding the methanation product flow in a first expander, with power production, thus obtaining a flow of expanded methanation products,
A3c2) cooling in a second exchanger, thus obtaining a flow of expanded and cooled methanation products, and
A3c3) separating a second condensed water vapor portion in a second separator, thus obtaining the flow of partially dehydrated methanation products.
